(19) United States
(12) Patent Application Publication
(10) Pub. No.: US 2011/0212491 A1
(43) Pub. Date: Sep. 1, 2011

(54) REACTION VESSEL

(75) Inventors: Martin Alan Lee, Wiltshire (GB); David James Squirrell, Wiltshire (GB); Ross Peter Jones, Cambridge (GB); Andrea Hamilton, Wiltshire (GB)

(73) Assignee: ENIGMA DIAGNOSTICS LIMITED, Wiltshire (GB)

(21) Appl. No.: 12/671,777

(22) PCT Filed: Aug. 1, 2008
(86) PCT No.: PCT/GB08/02629

§ 371 Date: Mar. 11, 2010

(30) Foreign Application Priority Data
Aug. 3, 2007 (GB) 0715169.9
Aug. 3, 2007 (GB) 0715170.7

(51) Int. Cl. C12P 19/34; C12M 1/00; C08G 61/10

(52) U.S. Cl. 435/91.2; 435/289.1; 435/304.1; 528/396

(57) ABSTRACT

A reaction vessel for carrying out a chemical or biochemical reaction, such as a polymerase chain reaction, said vessel having a coating of parylene or a derivative thereof, on at least the surface which contacts reactants.

The present invention relates to reaction vessels useful in chemical and biochemical reactions, in particular to chemical and biochemical reactions which are required to undergo controlled heating and/or cooling, in particular, vessels which are required to undergo thermal cycling, where a sequence of different temperatures are required.

A particular example of such a reaction are a number of nucleic acid amplification methods, for example ligase chain reaction (LCR), strand displacement amplification (SDA), transcription-mediated amplification (TMA), loop-mediated isothermal amplification (LAMP), rolling circle DNA amplification, multiplex ligation-dependent probe amplification (MLPA) and multiple displacement amplification, and in particular the polymerase chain reaction (PCR). As is well known, in this reaction, exponential amplification of nucleic acids is achieved by cycling the sample containing or suspected of containing the target nucleic acid through an iterative sequence of different temperatures in the presence of specifically designed primer sequences and polymerase enzymes able to extend those primer sequences. These temperatures represent the temperatures necessary for nucleic acid denaturation (and generally requires temperatures of about 95° C.), primer annealing (at a lower temperature for example at about 55° C.) and primer extension (which may require and intermediate temperature for example of about 74° C.).

There is frequently a need to obtain the results of a PCR reaction quickly, for example in cases of environmental contamination which may be the result of hostile activity. However, even in a clinical or diagnostic situation, the production of quick results can be helpful, in particular where patient compliance or return can be problematic.

Clearly, for fast PCR, the sample must be rapidly heated and cooled. This is facilitated in some instances by making the sample small to reduce its thermal mass and by minimising the distances over which heat must be transferred. The same considerations must be applied to the container of the sample.

Thus there may be particular advantages to using a reaction vessel which has a high thermal conductivity, to ensure that samples may be rapidly and controllably cycled through the requisite cycling regime. However, there is a problem in that many of the materials of this type are metallic in nature and they are not compatible with biological reactions. The reactive nature of the surface interferes with the biological molecules taking part in the reaction, and so may inhibit or even prevent a reaction taking place.

Thus in many cases, reaction vessels such as glass or specific polymers, are formed into reaction vessels and these are then subject to heating and cooling in a range of thermal cycling devices.

However, the precise nature of the vessel can impact on the reactions which are possible. Polypropylene is one of the most common plastics used for disposable laboratory ware and it is chosen because it is unaffected by aqueous solutions, it is biocompatible, readily moulded and has a melting point well above 100 degrees centigrade. It is the standard material used for manufacturing PCR tubes, but does have drawbacks in real-time PCR applications. These disadvantages are low thermal conductivity, which makes thermal cycling slow, and poor optical characteristics (low transmission), which interfere with fluorescence measurements where light scattering causes increased backgrounds.

For these reasons, glass is the preferred substrate used in as the reaction vessel in rapid real-time PCR instruments: the Roche “Light Cycler” and the Idaho Technology “RAPID” machines because it is optically superior to plastic materials.

There is, however, a problem associated with glass: reactions formulated for use in standard polypropylene tubes have to be re-formulated for use in glass tubes because of the surface properties of the glass and its interactions with reaction components such as proteins and inorganic ions. It is undesirable to make PCR reaction mixtures that will work optimally in both glass and polypropylene tubes proteins, as BSA (Bovine Serum Albumin) is required which causes issues with reagent handling and export controls.

Furthermore, such reaction vessels can have a significant thermal mass in their own right, and therefore, they reduce the efficiency of the process. Glass is much better in terms of thermal conductivity than polypropylene, but it is still less than optimum as compared to some other materials.

It has previously been described (Shin et al. J. Micromech. Microeng. 13 (2003) 768-774) how polydimethylsiloxane (PDMS)-based micro PCR chips may be prepared with parylene coatings.

The applicants have found however that this particular type of thin polymeric coating material is broadly compatible with chemical and biochemical reactions such as nucleic acid amplification reactions and in particular PCR, and therefore forms an ideal coating for reaction vessels in a variety of formats, where it may also provide significant advantages.

According to the present invention there is provided a reaction vessel, other than a PDMS microchip, for carrying out a chemical or biochemical reaction, said vessel having a coating of parylene or a derivative thereof, on at least the surface which contacts reactants.

Preferably the chemical or biochemical reaction is a nucleic acid amplification reaction.

The coating as described above has been found to be highly compatible with chemical of biochemical reactions, including those which use proteins or nucleic acids such as nucleic acid amplification reactions such as PCR, in a wide range of reaction vessels, including in particular, those which are not microfabricated. Thus, the coating may be applied to reaction vessels selected from tubes including capillary or other tubes as well as flasks and the like, which have a capacity in excess of 1 μl, for example in excess of 5 μl and suitably from 20 μl to 1 litre. The fact that the parylene coating is effective and robust enough to withstand the more vigorous handling and volumes of sample which such vessels are subject to, is quite unexpected. However, the versatility of the material and the enhancements in reaction efficiency which are detailed further below, are quite unexpected.

Thus the coating can be used to make a vessel compatible for use in such as reaction or enhance the compatibility thereof. By using vessels which are coated in this way, the need for reformulation of reaction mixtures such as PCR mixtures to take account of the nature of the vessel can be minimised or avoided. Furthermore, in some cases, increased efficiency of reaction such as PCR reaction can be achieved.

‘Parylene’ is a generic name applied to polyxylylene as for example as described in U.S. Pat. No. 3,343,754, the content of which is incorporated herein by reference.

Thus, a reference to parylene or derivatives include compounds which can be represented by the general formula (I)

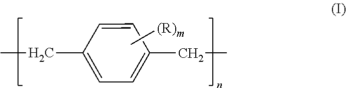

where is R is a substituent group, m is 0 or an integer of from 1 to 3 and n is sufficient for the compound to be a polymer.

Where m is greater than 1, each R group may be the same or different.

In one embodiment, m is 0.

Suitable substituent groups R include but are not limited to R¹, OR¹, SR¹, OC(O)R¹, C(O)OR¹, hydroxyl, halogen, nitro, nitrile, amine, carboxy or mercapto and where R¹ is any hydrocarbon group and where R¹ may be optionally substituted by one or more groups selected from hydroxyl, halogen, nitro, nitrile, amine or mercapto.

Suitable hydrocarbon groups include alkyl groups such as straight or branched chain C₁₋₁₀alkyl groups, alkenyl groups such as straight or branched C₂₋₁₀alkenyl groups, alkynyl groups such as straight or branched C₂₋₁₀alkynyl groups, aryl groups such as phenyl or napthyl, aralkyl groups such as aryl(C₁₋₁₀)alkyl for instance benzyl, C₃₋₁₀cycloalkyl, C₃₋₁₀cycloalkyl(C₁₋₁₀)alkyl, wherein any aryl or cycloalkyl groups may be optionally substituted with other hydrocarbon groups and in particular alkyl, alkenyl or alkynyl groups as described above.

Particular examples of groups R include alkyls such as methyl, ethyl, propyl, butyl or hexyl, which may be optionally substituted with hydroxy, halo or nitrile such as hydroxymethyl or hydroxyethyl, alkenyls such as vinyl, aryls in particular phenyl or napthyl which may be optionally substituted by halo or alkyl groups such as halophenyl or C₁₋₄alkylphenyl, alkoxy groups such as methoxy, ethoxy, propoxy, carboxy, carbomethoxy, carboethoxy, acetyl, propionyl or butyryl.

In particular, R is selected from halogen (particularly chlorine or bromine), methyl, trifluoromethyl ethyl, propyl, butyl, hexyl, phenyl, C₁₋₄alkylphenyl, naphthyl, cyclohexyl and benzyl.

Examples of such polymers are sold as “Parylene”. Particular variety of parylene which may be obtained include Parylene N (where m is 0), Parylene C (where m is 1 and R is chloro), Parylene F (where m is 1 and R is trifluoromethyl) and Parylene D (where m is 2 and each R is chloro).

Parylene is a particularly convenient polymeric material for providing a coating for reaction vessels, as it may be readily applied using a vapour deposition process. In this process a solid dimer of formula (II)

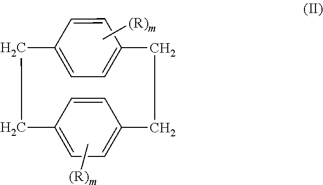

where R and m are as defined above, is placed into a suitable vaporisation chamber in solid form. When the chamber and heated under reduced pressure, for example to temperatures of about 150° C. at low pressure, for example of about 1 mmHg, the dimer vapourises. The vapour is then transferred into a pyrolysis chamber where the temperature is much higher, for example at about 650° C. and the pressure is for example of 0.5 mmHg. Pyrolysis occurs so as to cause the formation of a reactive monomeric species of formula (III).

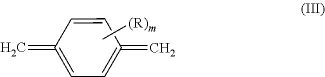

If this is allowed to pass into a further chamber containing the reaction vessel to be coated which is at ambient temperature, but also at low pressure, for example of 0.1 mmHg, polymerisation of the species (III) occurs on the surface of the object, so that a coating of the polymer of formula (I) above is produced.

The species (III) condenses on the surface in a polycrystalline fashion, providing a coating that is conformal and pinhole free.

This is important to ensure that any sample within the reaction vessel is isolated from the underlying wall.

Compared to liquid processes, the effects of gravity and surface tension are negligible so there is no bridging, thin-out, pinholes, puddling, run-off or sagging. And, since the process takes place at room temperature, there is no thermal or mechanical stress on the object.

Parylene is physically stable and chemically inert within its usable temperature range, which includes the temperatures at which PCR reactions are conducted. Parylene also provides excellent protection from moisture, corrosive vapours, solvents, airborne contaminants and other hostile environments.

It is widely used in the electronics industry to coat and protect electronic components, and is cost effective to apply. However, the applicants are the first to find that parylene is compatible with chemical or biochemical reactions and in particular with nucleic acid amplification reactions, such as the PCR reaction, even in a non-microfabricated environment.

The coating is suitably applied to the entire inner surface of the vessel, to ensure that reagents taking part in a reaction within the vessel do not come into contact with the underlying vessel walls. Conveniently however, the entire vessel is coated with parylene or a derivative thereof.

Because the parylene coating provides a safe and complete barrier, the nature of the underlying walls of the vessel may be of any convenient material. The selection of the material for the reaction vessel can be made on the basis of the desired properties of the vessel (e.g. strength, rigidity, transparency, thermal or electrical conductivity etc.) which may vary depending upon the particular chemical or biochemical reaction which is being conducted, and without regard for the compatibility of the material with the chemical or biochemical reaction occurring. Thus, they may comprise glass, polymers in particular rigid polymers, ceramic or even metallic materials such as metals or metal alloys, or any combinations or composites of these. Examples of polymers which may form the walls of the vessel include for example polyurethanes, polyethylene, polypropylene, polystyrene, polyesters, nylon, polycarbonates or polymethacrylate, for example polymethyl methacrylate (Perspex), as well as silicones.

For example, the reaction vessel may be a reaction vessel intended to carry out PCR, and therefore, be adapted to fit into a specific form of thermal cycling equipment. These may be heatable and/or coolable using a number of different technologies, including the use of fluid heaters and coolers such as air heaters and coolers in particular those heated by halogen bulbs, as described for example in U.S. Pat. No. 6,787,338 and WO2007/054747, the content of which is incorporated by reference, as well as in vessels using ECP as resistive heating elements, for example as described in WO 98/24548 and WO 2005/019836 as will be discussed further below. The vessels may also be used in more conventional devices such as solid block heaters that are heated by electrical elements. For cooling the apparatus may incorporate thermoelectric devices, compressor refrigerator technologies, forced air or cooling fluids as necessary.

Suitable vessels take various forms depending upon the nature of the thermal cycling equipment, including for example reaction tubes which are tapered inwards and the lower end, and may include caps, as well as elongate vessels such as capillary vessels. Vessels of the invention may take any of these forms.

In order to achieve rapid PCR however, there a number of examples of apparatus which utilize elongate reaction vessels.

In particular, the vessel is a capillary vessel or a flattened capillary vessel, where the length is selected to accommodate the volume of the sample and inner diameters are small. In particular, the inner diameter of a capillary tube is in the range of from 0.2 to 2 mm. The thickness of the wall is generally from about 0.1 to about 1.5 mm.

Examples of such vessels are described for example in WO2004/054715, U.S. Pat. No. 6,015,534 and WO 2005/019836, the content of which is incorporated herein by reference.

Where flattened tubes are used, they may be of a shape described in WO2006024879, the content of which is incorporated herein by reference. Specifically, such vessels have a width:depth ratio of about 2:1 or more, for example, of 3:1 or more. Typically the width of the vessels may be of the order of 1 mm or less for example 0.8 mm or less, whereas the depth is generally 0.5 mm or less, and suitably less than 0.3 mm. The vessels may be tapered.

Vessels of this shape form a particular embodiment of the invention.

In one PCR apparatus, electrically conducting polymer (ECP) is used as both the heating element and sometimes also the container (see WO 98/24548, the content of which is also incorporated herein by reference).

The ECP acts as a resistive heater and so it is required to be connected to an electrical supply by way of electrical connections. As an inevitable consequence of reducing the thermal mass of the sample and facilitating heat transfer into and out of it, the means of connecting and locating the ECP can have significant thermal effects upon it.

A particular problem associated with the use of elongate vessels is the formation of temperature gradients along the length as heat can be conducted both out from and in to the extremities of the vessel, in particular where for example, the extremities comprise electrical contacts, required to activate a resistive heater arranged along the tube. These gradients mean that the sample may not be uniformly thermally cycled and so the PCR reaction may be inhibited.

In PCR, it would be ideal to have all parts of the sample at the same controlled temperature all of the time. This is extremely difficult in a system that is being rapidly heated and cooled. In the capillary format, both radial and longitudinal temperature gradients are formed.

In accordance with the invention however, the walls of vessels for PCR may comprise a highly thermally conducting material such as a metallic material, so as to facilitate rapid transfer of heat into and out of the reactants undergoing the PCR, and also, reduce any thermal gradients which occur along the sample.

The use of such materials could not hitherto have been contemplated for PCR because of the inhibitory nature of the material on the reaction. Generally, metal walled vessels have not be used because the metals are generally chemically reactive in particular with biological molecules such as nucleic acids and proteins, and so the metal interferes with reagents in the vessel and so disrupts the reaction. Chemical and biochemical reactions such as nucleic acid amplification methods, for example the PCR are known to be sensitive to ionic milieu, especially in terms of metal ions. However, the reaction vessels as described above have a coating which isolates the reactants in the vessel from the material of the walls.

Coatings of parylene or derivatives are extremely thin and therefore have the benefit that they do not significantly add to the size or thermal mass of the vessel.

Thus vessels comprising a highly thermally conducting material most suitably comprise a material which has a thermal conductivity in excess 15 W/mK. Materials having this property will generally be metallic in nature, but certain polymers, in particular those known as “cool polymers” or polymers contaning thermally conducting compounds such as boron nitride as well as diamond, may have the desired level of thermal conductivity. In particular however, the highly thermally conducting material is metallic, which may be of any suitable metal or metal alloy including aluminium, iron, steel such as stainless steel, copper, lead, tin, brass or silver. In particular, the metallic layer comprises aluminium.

Furthermore, where the vessels of the invention comprise a metal or metal alloy, they may also be capable of being heated using for example induction methods. Apparatus used to heat vessels of the invention in this way will have the facility to heat the vessel by electromagnetic induction, for example by using a high-frequency alternating current (AC) to induce eddy currents within the metal. Resistance of the metal to these currents leads to Joule heating of the metal. Heat is also generated by magnetic hysteresis losses. For use in induction heating apparatus, it will be clear that the metallic layer within the vessel should be of a suitable material to allow it to be heated in this way, and so for example iron metallic layers may be preferred to say stainless steel or copper.

Where the vessels comprise a non-transparent material such as a metal or metal alloy, it may be desirable to ensure that at least a portion of the vessel is of a transparent material such as glass or a transparent polymer so that the contents of the reaction vessel can be optically monitored during the reaction. This is particularly helpful in the case of the use of the so-called “real-time” PCR reactions where optical signals, in particular fluorescent signals from signalling reagents added to the PCR reaction, produce a variable signal as the reaction progresses, so that the progress of the reaction can be monitored. Such monitoring gives rise to the option of quantifying the amount of target nucleic acid within the initial sample, so providing further information which may be of use, for example in diagnostics, in determining the seriousness of a particular condition.

The transparent portion may be appropriately arranged anywhere in the vessel, but in the case of elongate vessels as described above, it suitably forms the base portion. It is suitably coated with parylene or a derivative thereof in the same way as the rest of the vessel to ensure that constant levels of compatibility is maintained across the entire surface. In such cases however, the selection of a particular parylene derivative which is known to produce coatings which have good levels of transparency such as parylene C is suitably made. Furthermore the thickness of the coating should be kept as thin as possible, for example of 100 microns or less, for instance less than 50 microns, suitable less than 20 microns or more particularly less than 1 micron, to minimise deleterious effects of the coating on the optical properties of the transparent portion.

Such considerations apply in relation to any coating applied to a transparent vessel which is used in a manner in which the optical properties and in particular the transparency is important. Thus for example in glass PCR tubes, intended for use in real-time PCR systems where optical signals from within the vessel are monitored, are suitably coated with a layer of parylene which is thin enough not to significantly impair this function. For example, the coating may be 100 micron or less, for instance less than 20 microns or more particularly less than 1 micron, in thickness and/or the particular parylene derivative selected is one which is known to give high transparency levels.

The vessels may comprise composite walls in particular, where other materials are suitably layered thereon. A particular example of such as material is the ECP, which is arranged to act as a resistive heater, as described for example in WO 98/24548 and WO 2005/019836, and such vessels form a particular embodiment of the invention.

Reaction systems comprising combinations of reaction vessels as described above, and apparatus which is able to accommodate said reaction vessel, to allow the chemical or biochemical reaction to occur, such as apparatus which comprises a heating system adapted to controllably heat and cool said vessel, in particular using any of the methods discussed above, form a further aspect of the invention.

When the reaction vessels of the invention are utilised in combination with resistive heating elements, such as ECP, it is necessary to ensure that where the highly thermally conducting material of the vessel wall is also electrically conducting, such as a metallic layer, this is electrically insulated from the resistive heater in order to prevent short circuits etc. The applicants have found that it is possible to passivate the surface of a metal vessel wall so that it is electrically isolated from the ECP, but still in good thermal contact. For example in the case of an aluminium metallic layer, anodisation of any surface of the aluminium layer which is to be in contact with the resistive heater such as the ECP provides such insulation.

Alternatively, where the parylene layer covers the entire vessel, including the external surfaces, this contacts the ECP and provided an effective electrically insulating layer.

The ECP elements used in the vessels as resistive heating elements can be manufactured by various processes, but most a convenient process involves injection moulding of the polymer. However, in the process of injection moulding, the material tends to form an outer polymer-rich skin that may creates at least a partial electrically insulating barrier to any external means of making electrical contact.

In such cases, the applicants have found that it is helpful to break through the insulating skin and make electrical contact with the bulk material in order to increase the efficiency of the heating system.

Thus, in a particular embodiment, the reaction vessel as described above, is connectable to an electricity supply by means of barbed electrical contacts which pierce the surface of the electrically conducting polymer. These are suitably integral with the vessel.

Such barbed connectors may take various forms depending upon the particular configuration of the reaction vessel itself. In particular, where the vessel is of a generally tubular configuration, suitable barbed connectors may take the form of annular metal rings with inwardly projecting barbs or the like, similar in design to “Starlock Washers”.

The inwardly projecting barbs will cut through the insulating skin to make electrical contact with the bulk conducting material, as well as hold the ring in position. The outer portion will present a metallic surface for electrical interconnection, and so apparatus intended to accommodate the vessels will be configured appropriately.

Furthermore, the barbs provide effectively a scalloped edge which helps to reduce the size and therefore the thermal mass of the connectors and also, reduces the contact area with the ECP. This has the further advantage of further minimising heat exchange between the electrical connectors and the ECP, so further assisting in reducing unwanted thermal gradient formation.

The connectors and particularly the barbs thereof, are suitably constructed of a material which have high mechanical strength, so that the barbs can be sharpened to enhance the penetration ability. Whilst many metals are able to fulfil this function, a particularly suitable material for the connectors has been found to be stainless steel. This not only has the required mechanical strength and electrical conductivity, but also, it has a high corrosion resistance, at 16W/mK, a surprisingly low thermal conductivity compared to many other metals (cf Copper @399 W/mK, Aluminium @237 W/mK). By using this material, and by designing the connectors so that their area is as small as possible, helps to reduce unwanted thermal effects at the electrodes.

Connections of this type are cost effective to manufacture, which will be a particular advantage in cases where the proposed PCR vessel is to be a high volume disposable item.

Reaction vessels as described above and reaction systems comprising them can be used in chemical and biochemical reactions as required. Thus, in a further aspect, the invention provides a method for carrying out a nucleic acid amplification reaction which method comprising placing chemical or biochemical reagents into a reaction vessel as described above, and allowing the chemical or biochemical reaction to occur. In particular, the reaction is a polymerase chain reaction so the vessel will be heated and cooled appropriately. In particular, the vessel is positioned into apparatus specifically designed to hold it, and to heat and/or cool it as required. In particular, the apparatus comprises a thermal cycler as described above.

The invention will now be particularly described by way of example with reference to the accompanying diagrammatic drawings in which:

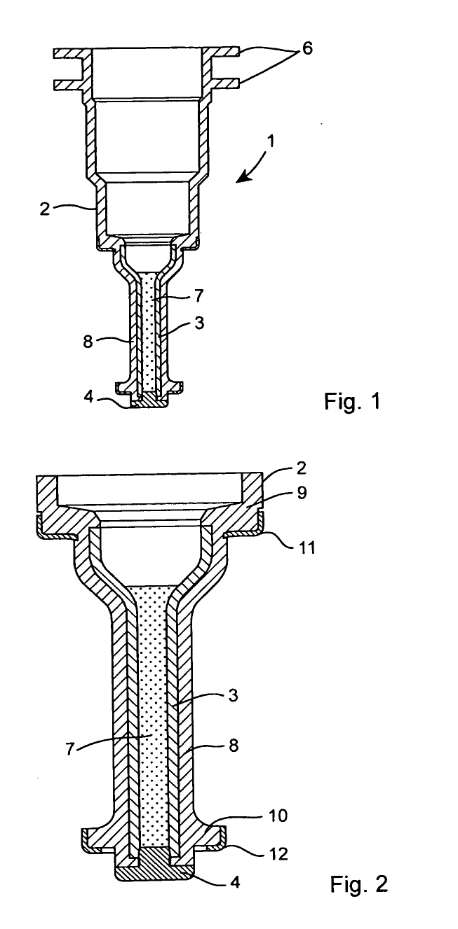

FIG. 1 shows a section through a reaction vessel according to the invention;

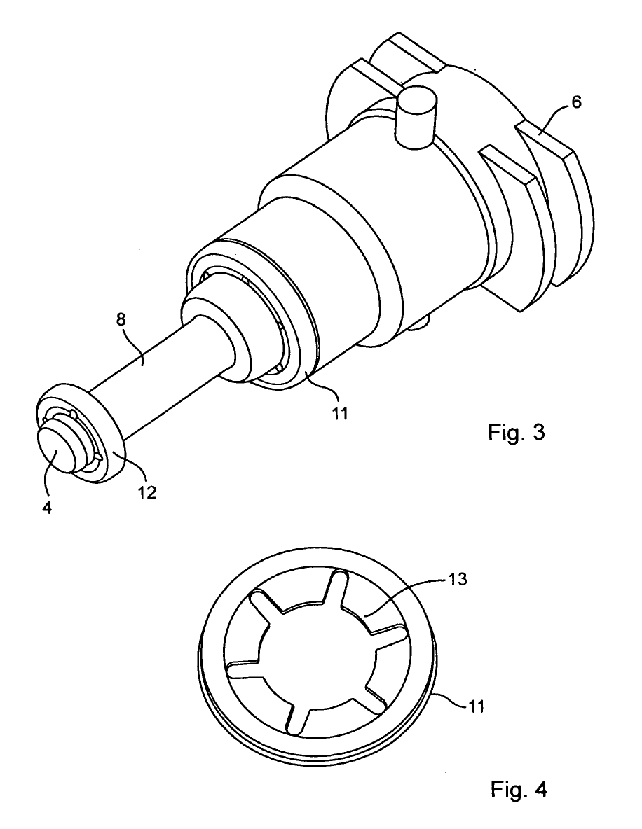

FIG. 2 shows an enlarged view of the portion of the reaction vessel shown in claim 1, which incorporates the elements of the invention;

FIG. 3 is a perspective end view of the reaction vessel of FIG. 1;

FIG. 4 shows an electrical connection used in the embodiment of FIG. 3; and

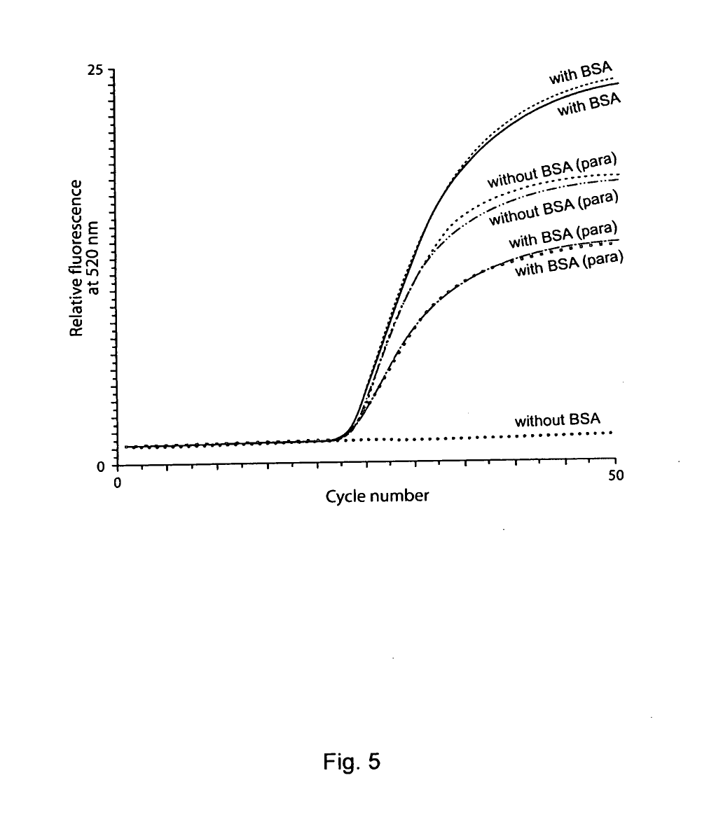

FIG. 5 shows a graph of results obtained when carrying out PCR in a range of parylene coated glass vessels.

### EXAMPLE 1

Metallic Reaction Vessels

The reaction vessel shown in FIG. 1 is of the same general type as those described in for example WO2005/019836, which is intended for use in an apparatus for conducting a PCR reaction.

The vessel comprises a plastics body (1) with an upper sample receiving portion (2) with a relatively wide mouth so that reagents can, with ease, be added. In the illustrated embodiment, the upper portion includes projecting flanges (6) which are able to interact with a lifting arm in an apparatus such as that described in WO 2005/019836 so as to allow the vessel to be moved in an apparatus adapted to carry out reactions automatically.

At the lower end of the sample receiving portion (2), the vessel terminates in an aluminium capillary tube (3) which is sealed at the lower end by a transparent seal (4), so as to form an elongate thin reaction vessel, which can contain relatively small sample (7) within the capillary section.

The aluminium capillary (3) is entirely coated with parylene, which forms a PCR compatible as well as an electrically insulating layer thereon.

An ECP layer (8) completely encases the aluminium capillary (5). Is it provided with upper and lower ridges (9, 10) respectively which can accommodate upper and lower annular electrical connectors (11, 12) respectively. Each electrical connector (11,12) is provided with a number of inwardly projecting barbs (13) (FIGS. 3 and 4) which are able to pierce the surface of the ECP to ensure that electrical contact is made with the body of the ECP.

In use, this particular vessel can be loaded with sample and PCR reagents, as described in WO 2005/019836. A prepared sample (7) to which has been added all the reagents necessary for carrying a PCR reaction is placed in the upper portion (2) and if necessary a cap (not shown) is placed over open end. The entire vessel is then centrifuged to force the sample (7) into the capillary tube (3) section of the vessel.

In an alternative embodiment however, the sample and the reagents may be loaded directly into the capillary tube (3) using a specifically designed fine tipped pipettor, and with accompanying close control of pipettor removal, as described and claimed in a copending British patent application of the applicants of even date to the present application.

The vessel is then suitably positioned in an apparatus able to accommodate it such that the connectors (11, 12) are connectable to an electrical supply such as that described in WO 2005/019836.

The connectors (11,12) are then connected to the electrical supply, which is controlled, suitably automatically, to pass current through the ECP layer (8) so it rapidly progresses through a series of heating and cooling cycles, ensuring that the sample is subjected to similar cycling conditions. The high thermal conductivity of the aluminium capillary tube (3) facilitates this. However, the parylene coating ensures that it does not inhibit the progress of the reaction, and that the current passing through the ECP layer (8) is not short circuited by contact with the aluminium. This will allow the sample (7) to be subjected to a PCR reaction.

Where a real-time monitoring system is included in the sample (7), the progress of the PCR can be monitored through the seal (4) using conventional methods.

As a result, rapid PCR can be achieved. The presence of thermal gradients within the vessel (1) and therefore the sample (7) is reduced by the measures taken, including in particular the presence of the aluminium coating layer (5) which acts as a thermal shunt to dissipate temperature gradients. Thus reliable and reproducible results may be achieved.

### EXAMPLE 2

Plastics Reaction Vessels

Polypropylene PCR tubes were coated with a thin film of parylene using conventional coating methods, in particular vapour deposition as described above.

PCR reactions were then carried out in these tubes as well as uncoated polypropylene PCR tubes using a RotorGene 3000 real-time PCR machine, and following the protocol set out below.
1) BVDV DNA was diluted 1:10 (10 μl stock and 90 μl water) twice to give 10⁻⁵ and 10⁻⁶ solutions.
2) A mastermix was prepared using the CAS1200 robot to the following recipe:

| | | |
|---|---|---|
| DNAse free water | 10.42 | μl |
| 500 mM Tris pH 8.8 | 2.00 | μl |
| 100 mM MgCl₂ | 0.60 | μl |
| 2 mM dUTP mix | 2.00 | μl |
| 10 μM A11 forward primer | 0.25 | μl |
| 10 μM A14 reverse primer | 0.25 | μl |
| 2 μM BVDV ½ probe | 2.00 | μl |
| Anti-Taq antibody | 0.32 | μl |
| Taq DNA polymerase (5 U/μl) | 0.16 | μl |

3) Transfer 18 μl of each type of mastermix polypropylene tubes (stock or Parylene-coated). Add 2 μl of either nucleic acid template (duplicates of each dilution) or water to appropriate tubes and then cap all tubes.
4) Load tubes into RotorGene and select appropriate run parameters for type of reaction as follows:
   95° C. denature step (30 seconds)
   95° C. for 20 seconds, 60° C. for 30 seconds (40 cycles amplification)

The results, which are the mean of two duplicate are set out in Table 1 below:

| | Process | Template and | Ct | Fluorescence |
|---|---|---|---|---|
| Tube format | (PCR/RT) | concn | value | signal |
| Native microfuge | PCR | 10-4 DNA | 23.40 | 0.71 |
| tube | | | | |
| D Parylene | PCR | 10-4 DNA | 23.99 | 0.63 |
| microfuge tube | | | | |
| N Parylene | PCR | 10-4 DNA | 23.82 | 0.66 |
| microfuge tube | | | | |
| Native microfuge | PCR | 10-6 DNA | 29.88 | 0.64 |
| tube | | | | |
| D Parylene | PCR | 10-6 DNA | 30.62 | 0.52 |
| microfuge tube | | | | |
| N Parylene | PCR | 10-6 DNA | 30.71 | 0.62 |
| microfuge tube | | | | |
| Native microfuge | RT | 10-3 RNA | 22.09 | 0.74 |
| tube | | | | |
| D Parylene | RT | 10-3 RNA | 22.97 | 0.64 |
| microfuge tube | | | | |
| N Parylene | RT | 10-3 RNA | 23.43 | 0.68 |
| microfuge tube | | | | |
| Native microfuge | RT | 10-4 RNA | 27.29 | 0.63 |
| tube | | | | |
| D Parylene | RT | 10-4 RNA | 28.29 | 0.57 |
| microfuge tube | | | | |
| N Parylene | RT | 10-4 RNA | 27.22 | 0.62 |
| microfuge tube | | | | |
| Native microfuge | RT | 10-5 RNA | 31.51 | 0.40 |
| tube | | | | |
| D Parylene | RT | 10-5 RNA | 32.84 | 0.32 |
| microfuge tube | | | | |
| N Parylene | RT | 10-5 RNA | 32.13 | 0.40 |
| microfuge tube | | | | |

The results show that in this experiment, fluorescence signal was slightly attenuated in the parylene tubes, but the difference is not significant. The Ct values for all of the samples are similar.

The use of parylene tubes had no adverse effect on PCR using the RotorGene. Ct values and signal were not affected.

The results showed that the performance of the PCR was very similar in the coated tubes as compared to the uncoated tubes. Therefore, it is clear that parylene forms a highly PCR compatible coating, and can provide performance equivalent to that of even polypropylene tubes. Parylene is therefore suitable for treating the surfaces of plastics such as polystyrene, polycarbonate or polymethylmethacrylate (Perspex) that have desirable structural and optical properties, but are poor in terms of biocompatibility.

### EXAMPLE 3

Glass Reaction Vessels

Glass capillary tubes useful in the Roche LightCycler® were also coated with parylene and were tested alongside uncoated capillaries in PCR reactions using the following protocal.
1) BG DNA was diluted 1:10, 1:100 and 1:1000 through serial dilutions (10 μl BG solution and 9091 water).
2) Two mastermixes were prepared using the CAS1200 robot—one which contained BSA and one which did not. The recipes for each mix are as follows:

| BSA mastermix | | |
|---|---|---|
| DNAse free water | 4.67 | μl |
| 500 mM Tris pH 8.8 | 2.00 | μl |
| 20 mg/ml BSA | 0.25 | μl |
| 100 mM MgCl₂ | 0.60 | μl |
| 2 mM dUTP mix | 2.00 | μl |
| 10 μM forward primer | 2.00 | μl |
| 10 μM reverse primer | 2.00 | μl |
| 2 μM BG acceptor probe | 2.00 | μl |
| 2 μM BG donor probe | 2.00 | μl |
| Anti-Taq antibody | 0.32 | μl |
| Taq DNA polymerase (5 U/μl) | 0.16 | μl |
| No BSA mastermix | | |
| DNAse free water | 4.92 | μl |
| 500 mM Tris pH 8.8 | 2.00 | μl |
| 100 mM MgCl₂ | 0.60 | μl |
| 2 mM dUTP mix | 2.00 | μl |
| 10 μM forward primer | 2.00 | μl |
| 10 μM reverse primer | 2.00 | μl |
| 2 μM BG acceptor probe | 2.00 | μl |
| 2 μM BG donor probe | 2.00 | μl |
| Anti-Taq antibody | 0.32 | μl |
| Taq DNA polymerase (5 U/μl) | 0.16 | μl |

3) Transfer 18 μl of each type of mastermix to LightCycler® capillary tubes. Add 2 μl of either DNA template (duplicates of each dilution) or water to appropriate tubes and then cap all tubes.
4) Briefly impulse all tubes in benchtop centrifuge
5) Load tubes into LightCycler® and select appropriate run parameters for type of reaction as follows:
   95° C. denature step (2 minutes)
   95° C. for 5 seconds, 65° C. for 30 seconds (50 cycles amplification)

The results obtained in this case are illustrated in FIG. 5.

Although the presence of parylene caused a slight reduction in the fluorescence signal as compared to the uncoated tubes, it allowed the reaction to proceed, even in the absence of BSA. When BSA was absent, normal capillary tubes showed no amplification as expected (BSA is used to stop reagents adhering to glass surfaces). However, samples in parylene coated vessels showed a signal which was of the same order as those in normal tubes with BSA.

Since the parylene coating is compatible with both polypropylene and glass tubes, it is clear that it may be used to coat glass vessels in order to make them compatible with PCR reaction mixes that have been formulated for standard plastics tubes, without having adverse consequences for the optical or thermal properties of the glass tube format. This should allow for reaction mixes to be standardized and the reformulation and the need for additives which are sometimes required to allow PCR reactions to proceed in specifically glass vessels, can be avoided.

In order to minimize the optical effects on the glass tube, a parylene coating of less than 20 micron thickness, for example less than 1 micron is particularly suitable.

1. A reaction vessel other than a polydimethylsiloxane microchip for carrying out a nucleic acid amplification reaction, the reaction vessel comprising a coating of parylene or a derivative thereof, on at least a surface of the reaction vessel which contacts reactants.

2. The reaction vessel according to claim 1 which is a tube or flask.

3. The reaction vessel according to claim 1 wherein the coating comprises a compound of formula (I) 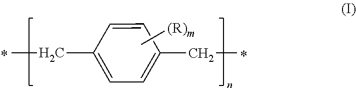
wherein
R is selected from R¹, OR¹, SR¹, OC(O)R¹, C(O)OR¹, hydroxyl, halogen, nitro, nitrile, amine, carboxy or and mercapto;
R¹ is hydrocarbon or hydrocarbon substituted by one or more groups selected from hydroxyl, halogen, nitro, nitrile, amine and mercapto;
m is 0 or an integer of from 1 to and
n is sufficient for the compound to be a polymer.

4. The reaction vessel according to claim 3 wherein the coating comprises a compound of formula (I) selected from
wherein m is 0;
wherein m is 1 and R is chlorol;
wherein m is 1 and R is trifluoromethyl; and
wherein m is 2 and each R is chloro.

5. A reaction vessel according to claim 1 wherein the coating is suitably applied to an entire inner surface of the reaction vessel.

6. The reaction vessel according to claim 1 wherein the walls of the reaction vessel comprise glass, polymer, ceramic or metallic materials.

7. The reaction vessel according to claim 6 wherein the walls of the reaction vessel comprise polypropylene, polymethacrylate, polycarbonate or polystyrene.

8. The reaction vessel according to claim 6 wherein the walls of the reaction vessel comprise glass.

9. The reaction vessel according to claim 8 wherein a thickness of the coating is less than 50 microns.

10. The reaction vessel according to claim 1 wherein the reaction vessel is a polymerase chain reaction vessel, which is adapted to fit into a thermal cycler.

11. The reaction vessel according to claim 10 which is in the form of a tapered reaction tube.

12. The reaction vessel according to claim 10 wherein the reaction vessel is a capillary vessel or a flattened capillary vessel.

13. The reaction vessel according to claim 10 wherein the walls of the reaction vessel comprise a highly thermally conducting material.

14. The reaction vessel according to claim 13 wherein the highly thermally conducting material is a metal or metal alloy.

15. The reaction vessel according to claim 14 wherein the metal or metal alloy is aluminium, iron, steel including stainless steel, lead, tin, brass, silver or copper.

16. The reaction vessel according to claim 1 which comprises an electrically conducting polymer layer, arranged to act as a resistive heater, thereon.

17. The reaction vessel according to claim 16 which further comprises one or more barbed electrical connectors which penetrate a surface of the electrically conducting polymer.

18. The reaction vessel according to claim 1 wherein the nucleic acid amplification reaction is a polymerase chain reaction.

19. A method of carrying out a nucleic acid amplification reaction which comprises a using the reaction vessel according claim 1 to carry out the nucleic acid amplification reaction.

20. The method according to claim 19 wherein the reaction is a polymerase chain reaction.

21. A method for producing a reaction vessel which comprises depositing a layer of parylene or a derivative thereof, onto the reaction vessel according to claim 1.

22. A reaction system comprising a reaction vessel according to claim 1 and an apparatus adapted to hold the reaction vessel and to allow a nucleic acid amplification reaction to be carried out.

23. The reaction system according to claim 22 wherein the apparatus is a thermal cycler.
